(19) United States
(12) Patent Application Publication
(10) Pub. No.: US 2007/0088174 A1
(43) Pub. Date: Apr. 19, 2007

(54) Synthesis of phenoxyacetic acid derivatives

(75) Inventors: Eric Winter, Gau-Algesheim (DE); Carsten Reichel, Rheinboellen (DE); Dieter Gutheil, Bad Kreuznach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/253,311

(22) Filed: Oct. 19, 2005

(51) Int. Cl. C07C 229/64

(52) U.S. Cl. 560/37; 562/442

(57) ABSTRACT

The present invention relates to an improved process for the preparation of substituted 2-(4-carbonylmethoxy-optionally 2,5-disubstituted-phenyl)-acetaldehydes, in particular 2-(4-alkoxycarbonylmethoxy-optionally 2,5-disubstituted-phenyl)-acetaldehydes and their use in the synthesis of optionally substituted 2-[4-[2-[[-2-hydroxy-2-(4-hydroxyphenyl)-1-methylethyl]-amino]ethyl]-optionally 2,5-disubstituted-phenoxy]acetic acid derivatives or the salts thereof, which may be used as pharmaceutically active substances.

### BACKGROUND OF THE INVENTION

The present invention relates to an improved process for the preparation of substituted 2-(4-carbonylmethoxy-2,5-disubstituted-phenyloxy)-acetaldehydes in industrial scale. In particular 2-(4-alkoxycarbonylmethoxy-disubstituted-phenyloxy)-acetaldehydes and their use in the industrial manufacture of optionally substituted 2-[4-[2-[[2-hydroxy-2-(4-hydroxyphenyl)-1-methylethyl]-amino]ethyl]-2,5-disubstituted phenoxy]acetic acid derivatives or the salts thereof is claimed. In particular the present inventions concerns the synthesis of (−)-Ethyl-2-[4-(2-{[(1S,2R)-2-hydroxy-2-(4-hydroxyphenyl)-1-methylethyl]amino}ethyl)-2,5-dimethylphenyloxy]acetate and (−)-2-[4-(2-{[(1S,2R)-2-hydroxy-2-(4-hydroxyphenyl)-1-methylethyl]amino}ethyl)-2,5-di-methylphenyloxy]acetic acid, salts thereof respectively, which may be used as pharmaceutically active substances.

The subject of the present invention is the synthesis in industrial scale of 2-[4-[2-[[2-hydroxy-2-(4-hydroxyphenyl)-1-methylethyl]-amino]ethyl]-2,5-disubstitutedphenoxy]acetic acid derivatives, which are represented by following formula (I): 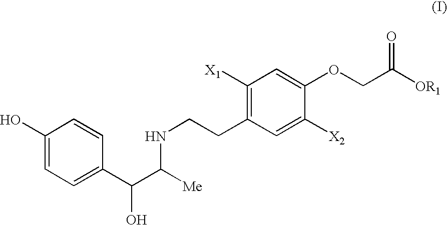
R₁ is H, branched or unbranched C₁₋₆-alkyl, or optionally substituted benzyl, and preferably branched or unbranched C₁₋₆-alkyl or optionally substituted benzyl. If R₁ is C₁₋₆-alkyl, R₁ is preferably methyl, ethyl, or propyl, more preferably ethyl or propyl, and most preferably ethyl. After manufacture of the compounds according to formula (I), R₁ can be turned into H (if it was not H before) by hydrolysis, as known in the art, or into NH₂, NHC₁₋₆-alkyl, or N(C₁₋₆-alkyl )2, with C₁₋₆-alkyl being as defined above, by the methods as known in the art. X₁ or X₂ are, independently from each other, hydrogen, halogen, or branched or unbranched C₁₋₆-alkyl. If X₁ and/or X₂ is halogen, X₁ and/or X₂ is preferably F, Cl, or Br. If X₁ and/or X₂ is C₁₋₆-alkyl, X₁ or X₂ is preferably methyl, ethyl, or propyl, more preferably methyl or ethyl, and most preferably methyl.

In the context of the present description the term “optionally substituted benzyl” shall mean that the aromatic ring system of the benzyl group may be substituted by: branched or unbranched C₁₋₆-alkyl and/or C₁₋₆-alkoxyl, which may be optionally substituted by a halo selected from the group of fluoro, chloro, bromo, and iodo, and which particularly preferably are methyl, ethyl, and/or trifluormethyl; 1 to 6 halogens that are independently selected from the group of fluoro, chloro, bromo, and iodo; —CN; nitro; hydroxy; and amino, which may be optionally substituted by C₁₋₆-alkyl, and which is dimethylamino or diethylamino in particular.

The preferred compounds to manufacture according to the present invention are

1) X₁=Br; X₂=H; R₁=H

2) X₁=Cl; X₂=H; R₁=H

3) X₁=Cl; X₂=Cl; R₁=H

4) X₁=H; X₂=H; R₁=H

5) X₁=Cl; X₂=H; R₁=H

7) X₁=Cl; X₁=Cl; R₁=Et

8) X₁=Me; X₁=Me; R₁=Et

9) X₁=Me; X₁=Me; R₁=H.

Preferred stereospecific details are given in formula (II): 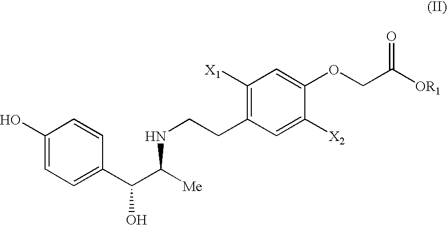
wherein X₁, X₂ and R₁ are defined as above, with all preferences as above (in particular compounds 1 to 9).

The claimed compounds according to formula (I) or (II) can be named as R₁—(−)-2-[4-[2-[[(1S, 2R)-2-hydroxy-2-(4-hydroxyphenyl)-1-methylethyl]-amino]ethyl]-2-X₂,5-X₁ phenoxy]acetates, the corresponding acetic acid derivative (R₁=H), or a pharmacologically acceptable salts of any of them.

The compounds of formula (I) are known from EP 1 095 932, JP-2002-338513 and other publications. They have a β₃-adrenergic receptor-stimulating effect (β₃-adrenergic receptor agonists) and are interesting as agents for preventing or treating obesity, adiposis, hyperglycemia, diseases caused by intestinal hypermotility, diseases caused by intestinal hyperkinesia, pollakiuria, urinary incontinence, depresssion, diseases caused by biliary calculi or hypermotility of the biliary tract, and cholelithiasis. Among the most preferred indication areas are urinary incontinence, be it in form of overactive bladder, stress urinary incontinence, urge urinary incontinence, or mixed forms thereof.

For the sake of clarity and completeness a certain terminology will be used hereinafter. The compounds according to general formula (I) shall include the embodiment described, expressis verbis, as well as all chemical or pharmacological equivalents. The compounds can be turned into pharmacologically acceptable salts thereof. Examples of pharmaceutically active salts for each of the compounds that are the subject of this description include, without being restricted thereto, salts that are prepared from pharmaceutically acceptable acids, including organic and inorganic acids. Suitable pharmaceutically acceptable acids include acetic acid, benzenesulphonic acid (besylate), benzoic acid, p-bromophenylsulphonic acid, camphorsulphonic acid, carbonic acid, citric acid, ethanesulphonic acid, fumaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid, and the like.

Methods for preparing compounds according to formula (I) are disclosed in EP 1 095 932 and JP-2002-338513. The synthetic route disclosed in JP-2002-338513 requires 5 steps starting from a compound of formula (III): 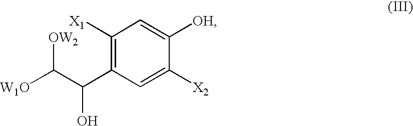
wherein X₁ and X₂ are both methyl and W₁ and W₂ are both alkyl groups. Up to the recrystallized end product of formula (I), three isolated intermediates are passed, which are acetals or semiacetals, and which have been found to be sensitive to certain physical properties like temperature, and which make a technical manufacturing process in industrial standard complex and difficult.

Compounds of formula (III) are available over the method described in JP-2002-338513 for example. In particular, example 1 of JP-2002-338513 describes the synthesis of the compound of formula (III), wherein X₁ and X₂ as well as W₂ and W₃ are methyl, for which the present synthetic rout can be applied as well.

### SUMMARY OF THE INVENTION

Accordingly, it is one objective of the present invention to improve the synthesis known from the prior art.

It is another aspect of the present invention to present a manufacturing process for to produce a compound of formula (I) in high amounts in industrial standard.

It is another objective of the present invention to present a manufacturing process for to produce a compound of formula (I) which passes stable with long shelf-life time.

Another objective is to create a manufacturing process with good manufacturing properties.

It is another objective of the present invention to create a manufacturing process with a reduced number of steps and finally with optimised yields of the products.

The objectives are met by the method according to the invention, because the new route comprises only 3 steps from the compound of formula (III) up to the end product (formula (I)) and creates stable intermediates, for which storage is uncomplicated.

Additionally, the inventive synthetic routs allow the production of a compound according to formula (I) in high amounts and in industrial standard.

Scheme 1 gives an overview about the method of preparation according to the invention:

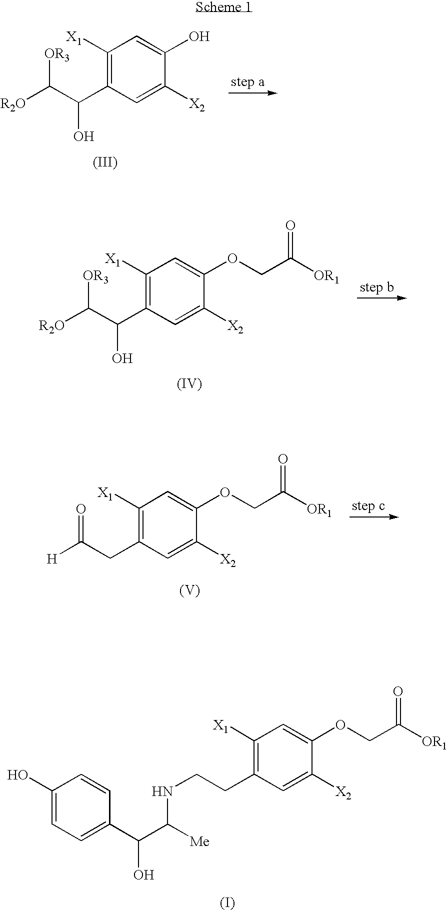

In the above scheme:

R₁ preferably is branched or unbranched C₁₋₆-alkyl or H; preferably it is C₁₋₆-alkyl, among which methyl, ethyl and propyl are preferred. More preferred are propyl and ethyl and most preferred is ethyl;

R₂ and R₃ are independently branched or unbranched C₁₋₆-alkyl, or both R₂ and R₃ together are a 5- or 6-membered saturated ring system, such as 1,3-Dioxanyl or 1,3 Dioxolanyl. R₂ and R₃ are each preferably a C₁₋₆-alkyl, among which methyl, ethyl, or propyl are preferred, methyl and ethyl are more preferred, and methyl is most preferred; and

X₁ or X₂ are independently as defined above and preferably C₁₋₆-alkyl, among which methyl, ethyl, and propyl are preferred, methyl and ethyl are more preferred, and methyl is most preferred.

It will be appreciated that, in the course of the synthetic route according to the present invention, there is made use of a new class of compounds as key intermediates, which key intermediate compounds are also subject of the present invention. Such compounds are 2-(4-(substituted carbonyl)methoxy-2,5-disubstituted-phenyl)-acetaldehydes, which are another objective of the present invention. They are represented by formula (V): 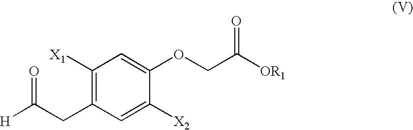
wherein R₁ and X₁ and X₂ are as defined above.

Another aspect of the invention is the synthesis of compounds of formula (I) starting from compounds of formula (IV) (see Step B hereinbelow).

The different steps of the method according to the invention as outlined in scheme 1 may be carried out according to procedures known, per se, particularly according to the following procedures. For the sake of clarity it shall be pointed out that the building blocks and intermediates used as herein described may be varied according to the knowledge of the state of the art to finally achieve the same final product. Such modifications include but are not limited to masking one or more groups which are not intended in the particular step by the reversible introduction of an appropriate protecting group or a reversible transformation of said group and the like. The present invention refers to such alternatives and equivalents which for the skilled person in the art are easy to be achieved.

### DETAILED DESCRIPTION OF THE INVENTION

Step A:

The phenoxyacetic acid ester derivatives represented by the above general formula (IV) can be prepared by reacting a phenol derivative of general formula (III) with a compound of formula (VI):
ZCH₂CO₂R₁ (VI)
wherein Z represents a substitution group, such as a halogen atom (e.g., chlorine or bromine), tosylate, or CO2R₁, wherein R₁ is as defined above.

The preferred reaction conditions comprise an inert solvent, a temperature of 0 to 100° C., and a reaction time of 1 to 24 hours. In case of Z being a halogen, catalytic amounts of sodium iodide may be added to the reaction mixture.

The inert solvents that are suitable for this reaction, include, for example, ethers, such as tetrahydrofuran, ketones, such as acetone and methyl ethyl ketone, acetonitrile, N,N-dimethylformamide, N,N-dimethylacetamide, and their mixtures. The mixtures may contain two or more of the above-mentioned solvents. As base, inorganic or organic bases may be used. As example of inorganic bases are named: sodium or potassium hydroxide, sodium carbonate, potassium carbonate, and cesium carbonate, as examples of organic bases are named triethylamine or ethyl-diisopropylamine. The reaction also may be carried out under phase transfer conditions. Usually 1 to 5 equivalents of the compound of general formula (VI) and of the base are used per equivalent of the compound of general formula (III). As for the molar ratio of the compound of formula (VI) and of the base, although they are usually used in equimolar amounts, either of them may be used in excess.

After the completion of the reaction, the reaction product is extracted and concentrated by ordinary methods to obtain the desired phenoxyacetic acid ester derivative of the general formula (IV). The phenoxyacetic acid ester derivative (IV) may be purified before entering the subsequent step, but it is also possible to use it in the next step without purification.

In a preferred variation of the step A, the compound of formula (III) is reacted with about 1.2 equivalents of a compound of the above general formula (VI), wherein Z is a bromine atom, in the presence of about 1.3 equivalents of potassium carbonate and catalytic amounts of sodium iodide in acetone for about 3 hours under reflux to yield the compound of formula (IV).

Step B:

The phenoxyacetic acid ester derivatives of above general formula (IV) are then transformed into the aldehydes of general formula (V) by transforming the acetal into the aldehyde while simultaneous and/or subsequent reduction of the hydroxyl group.

The reduction of the hydroxyl group may be performed by transforming the hydroxyl group of the compound of formula (IV) into a leaving group, for example, by reacting the compound of general formula (IV) with a trialkylhalosilane, such as trimethylchlorosilane, methyidiphenylchlorosilane, tert-butyl-dimethylchlorosilane, tert-butyl-diphenylchlorosilane, or the like, to give the corresponding trialkylsilyloxy derivative. Such a silyl-group can be cleaved subsequently under reductive conditions. For the silylation, 2 to 5 equivalents of the trialkylhalosilane can be used, with the use of about 3.1 equivalents being preferred. Additionally, sodium iodide may be added in an amount similar to that of the trialkylhalosilane. Suitable solvents for the reaction include, but are not limited to, acetonitrile, which is preferred. The reaction is usually carried out at a temperature between −50 and +25° C., preferably between −40 and 0° C., and most preferably between −15 and −25° C., particularly at about −20° C. The reaction time may vary between 1 and 24 hours, but often 1-3 hours, and, in particular, about 2 hours, will be enough for completion of the reaction. The reaction mixture then may be washed with aqueous solutions of sodium acetate and sodium thiosulfate. After the completion of the reaction, the reaction product is extracted and concentrated by ordinary methods.

Before the removal of the dimethoxy group, the residue so obtained may be optionally charcoaled using a suitable solvent, such as tetrahydrofuran, dioxane, methanol, ethanol, toluene, or the like. The purified solution thus obtained, or the unpurified residue dissolved in one of the solvents listed as suitable for charcoaling, is then treated with water and oxalic acid, perchloro acid, sulphuric acid, hydrochloric acid, and/or p-toluene sulfonic acid for several hours at room temperature. In general, 1-10 equivalents of oxalic acid are used, wherein about 3.4 equivalents is preferred. The work up is done by ordinary methods.

Step C:

For to make the final product, the aldehyde of general formula (V) is reacted with the corresponding amine, preferably 4-hydroxy-norephedrine (HNE), which has the following formula:

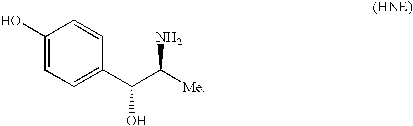

In alternative routes, an enatiomer or diasteromer of the compound can be used, as well as a racemic form, whereby it is noted that two chiral centres are present in HNE. In case of a racemic form of HNE, racemic separation may be performed in a subsequent step to complete the manufacture of the preferred final product of (1S,2R) configuration. It is also possible to protect the OH-group(s) by an appropriate protecting group, such as disclosed in the state of the art.

The coupling reaction of the aldehyde (V) and the amine (HNE preferably) is done in the presence of a reducing agent in an inert solvent. The temperature is preferably kept between −20 and 60° C. until completion or stop of the reaction. The reaction time usually is between 1 and 48 hours. Suitable reducing agents include alkali metal borohydrides, such as NaBH₄, NaCNBH₃, NaBH(OAc)₃, and NaBH(OMe)₃, and borane compounds, such as BH₃•pyridine and BH₃•N,N-diethylamine. If necessary, they can be used in the presence of an acid, such as acetic acid, p-toluenesulfonic acid, methanesulfonic acid, sulphuric acid, or hydrochloric acid, or a base, such as triethylamine. Furthermore, a catalytic amount of a metallic catalyst, such as 5% to 10% palladium carbon, Raney nickel, platinum oxide, palladium black or 10% platinum carbon (sulphur-poisoned), can be used in a hydrogen atmosphere. When an alkali metal borohydride of a borane is used as the reducing agent, the amount thereof is suitably selected in the range of 0.5 to 5 equivalents per equivalent of the aldehyde of formula (V). The inert solvents which can be used for this reaction include, for example, ethers such as tetrahydrofuran, 1,2-dimethoxyethane, and dioxane; halogenated hydrocarbons, such as methylene chloride and 1,2-dichloro-ethane; organic carboxylic acids, such as acetic acid; hydrocarbons, such as toluene; alcohols, such as methanol and ethanol; and acetonitrile. These solvents can be used either alone or in the form of a mixture of two or more of them. After the completion of the reaction, the insoluble matter is removed if necessary and the product is extracted and concentrated by ordinary methods to obtain the desired phenoxyacetic acid derivative of formula (I).

The preferred reducing agent is Pd/C under a hydrogen atmosphere, particularly at a concentration of 10%. Tetrahydrofuran is preferred as a solvent.

The phenoxyacetic acid derivative of formula (I) can be converted into a physiologically acceptable salt thereof, if desired, by an ordinary method. The salts include acid addition salts thereof with inorganic acids, such as hydrochloric acid, hydrobromic acid, hydroiodic acid, sulphuric acid, and phosphoric acid, as well as acid addition salts thereof with organic acids, such as formic acid, acetic acid, methanesulfonic acid, benzenesulfonic acid, p-toluenesulfonic acid, propionic acid, citric acid, succinic acid, tartaric acid, fumaric acid, butyric acid, oxalic acid, malonic acid, maleic acid, lactic acid, malic acid, carbonic acid, glutamic acid, and aspartic acid. By preference, the hydrochloric acid addition salt of the compound of formula (I) is prepared.

Optional Step D:

Optionally, for further purification, the compound of general formula (I), or its acid addition salt thus obtained, may be recrystallized using suitable solvents. Such suitable solvents include alcohols, such as methanol, ethanol, butanol, t-butanol, or isopropanol, and ethers, such as methyl tert-butyl ether or diethyl ether.

In a preferred variation of the Step D, the hydrochloride of the compound of formula (I) is recrystallized from a mixture containing 40 vol-% of ethanol and 60 vol-% of methyl tert-butyl ether. The isolated crystals are washed with ice-cold mixtures of ethanol and methyl tert-butyl ether with a even larger amount of methyl tert-butyl ether than in the mother liquor and subsequently with methyl tert-butyl ether alone.

Optional Steps E or F:

Yet another optional step is transforming the product according to the step C or D into a salt form, if it is not already the wished salt. To do so, reference is made to the prior art, in particular, as disclosed above.

As mentioned above, the compounds according to formula (I) or (II) with R₁ being alkyl can optionally be turned into the free acid by hydrolyzation methods or into an amide by amination methods as known in the art.

The advantage of the present invention over the prior art in particular are:

significantly improved overall yield in a chemical process of industrial scale;

avoidance of instable intermediates such as semi-acetals;

the process is shortened by one step; and

better capacity-time yield. The term capacity refers to the capacity of the reaction vessel in cubic meter, the term time to the reaction time needed to manufacture 1 kg of substance.

The major improvement is a better overall yield which is of high importance in particular for a chemical process of industrial scale.

The following examples are intended to illustrate the invention in greater detail:

### EXAMPLE 1

### Ethyl 2-[4-(2,2-dimethoxy-1-hydroxyethyl)-2,5-dimethylphenoxy]acetate

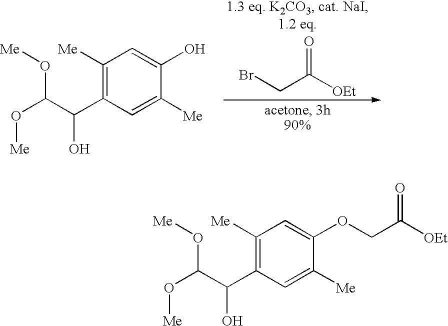

4-(2,2-Dimethoxy-1-hydroxyethyl)-2,5-dimethylphenol (20.0 g, 88.3 mmol, 1.0 eq.), K₂CO₃ (15.9 g, 115 mmol, 1.3 eq.), ethyl bromoacetate (17.7 g, 106 mmol, 1.2 eq.) and Nal (cat. amount) are mixed in acetone (20 ml) at room temperature. The suspension is stirred and refluxed for 3 h. After adding triethylamine (5 ml, 35 mmol, 0.4 eq.) the mixture is diluted with toluene (150 ml) and washed with aq. NaOH (0.5 M, 100 ml) and water (100 ml). The organic phase is concentrated to an oily residue and cyclohexane (400 ml) is added at 55° C. After cooling down to 0° C., the white crystals are filtered off, washed with cyclohexane (60 ml) and dried at 45° C i.v.

Yield: 24.8 g, 79.5 mmol, 90%

Melting point: 83° C.

### EXAMPLE 2

### 2-(4-Ethoxycarbonylmethoxy-2,5-dimethyl-phenyl)-acetaldehyde

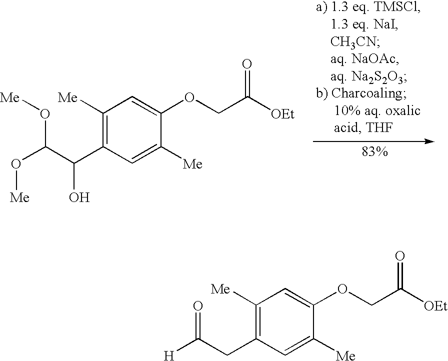

Nal (29.8 9,197 mmol, 3.1 eq.) and trimethylchlorosilane (21.6 g, 197 mmol, 3.1 eq.) are stirred at 5° C. in acetonitrile (50 ml) for 15 min., then the suspension is cooled down to −20° C. A solution of ethyl 2-[4-(2,2-dimethoxy-1-hydroxyethyl)-2,5-dimethylphenoxy]acetate (20.0 g, 63.0 mmol, 1.0 eq.) in acetonitrile (50 ml) is added and the mixture is stirred for 2 h. For workup, aq. NaHCO₃ (150 ml) and sat. aq. Na₂S₂O₃ (90 ml) are added and the mixture is diluted with toluene (140 ml) and warmed up to 5° C. The organic phase is separated and washed with aq. Na₂S₂O₃ (40 ml) and water (2×40 ml). The solvent of the organic phase is distilled off i. v. completely and the oily residue is diluted with THF (50 ml). The solution is charcoaled and after filtration the organic phase is treated with water (170 ml) and oxalic acid (20.0 g, 218 mmol, 3.4 eq.). The reaction is complete after 3.5 h and toluene (140 ml) is added. After phase separation the organic phase is washed with water (2×40 ml), aq. NaHCO₃ (40 ml) and again water (2×40 ml). Finally, the crude product is concentrated.

Yield: 13.1 g, 52.3 mmol, 83%

### EXAMPLE 3

### Ethyl (−)-2-[4-[2-[[(1S,2R)-2-hydroxy-2-(4-hydroxyphenyl)-1-methylethyl]-amino]-ethyl]-2,5-dimethylphenoxy]acetate hydrochloride

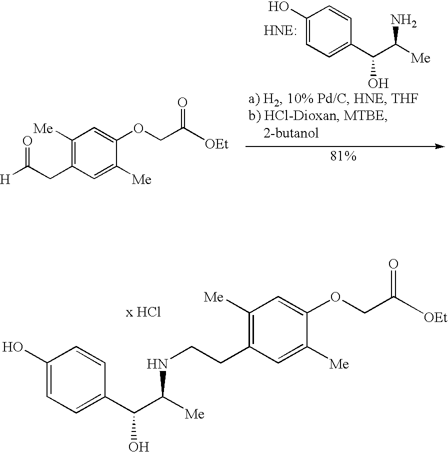

2-(4-Ethoxycarbonylmethoxy-2,5-dimethyl-phenyl)-acetaldehyde (30.0 g, 120 mmol, 1.1 eq.), 4-hydroxy-norephedrine (18.4 g, 110 mmol, 1.0 eq.) and Pd/C (10% Pd, 50% water, 7.6 g) are mixed in tetrahydrofuran (THF) (300 ml) at room temperature, then H₂ is bubbled through the suspension until the reaction is finished. After filtration, concentration and washing with toluene (300 ml) the organic phase is washed with water (3×150 ml). The solution is concentrated and 2-butanol (60 ml) is added. At 70° C. HCl (˜1.5 mol/l in 1,4-dioxane, 60 ml, 0.85 eq.) is dropped to the reaction mixture, and the suspension is cooled down to 50° C. Then, methyl tert-butyl ether (300 ml) is added slowly. The crystals are stirred overnight, filtered off, washed with ethanol/methyl tert-butyl ether (1:5, 60 ml) and methyl tert-butyl ether (60 ml) and dried at 75° C. i. v.

Yield: 39.0 g, 89.0 mmol, 81%.

Melting point: 176° C.

### EXAMPLE 4

### Recrystallisation of Ethyl (−)-2-[4-[2-[[(1S,2R)-2-hydroxy-2-(4-hydroxyphenyl)-1-methylethyl]-amino]ethyl]-2,5-dimethylphenoxy]acetate hydrochloride

Ethyl (−)-2-[4-[2-[[(1S,2R)-2-hydroxy-2-(4-hydroxyphenyl)-1-methylethyl]-amino]-ethyl]-2,5-dimethylphenoxy]acetate hydrochloride (e.g. from example 3) (20.0 g, 45.6 mmol) is solved in ethanol (110 ml) at 78° C. The clear solution is cooled to 58° C. and methyl tert-butyl ether (172 ml) is added slowly. After cooling down to 0° C. the crystals are filtered off, washed with ice-cold ethanol/methyl tert-butyl ether (1:5, 50 ml) and methyl tert-butyl ether (50 ml). The white crystals are dried at 70° C. i.v.

Yield: 16.6 g, 37.9 mmol, 83%.

Melting point: 196-197° C.

Other compounds can be made accordingly. Preferred is the synthesis of the compounds according the examples.

1. A method for the preparation of compounds of formula (I) 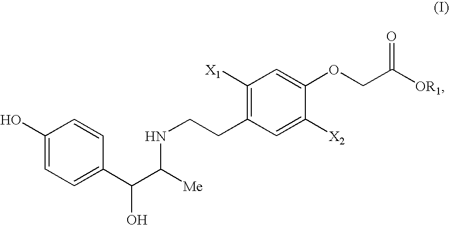
or a salt thereof, wherein R₁ is H, branched or unbranched C₁₋₆-alkyl, or optionally substituted benzyl, and X₁ and X₂ are independently from each other H, halogen, or branched or unbranched C₁₋₆-alkyl, the method comprising:
A. reacting
i. a compound of formula (III) 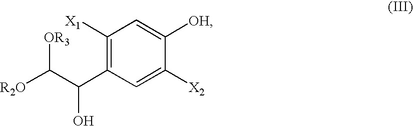
wherein X₁ or X₂ are as defined above, and wherein R₂ and R₃ are independently of each other branched or unbranched C₁₋₆-alkyl, or both R₂ and R₃ together are a 5- or 6-membered saturated ring system, and
ii. a compound of formula (VI)
ZCH₂CO₂R₁ (VI),
wherein Z represents a substitution group and R₁ is as defined above,
B. transforming the compound obtained according the aforementioned step, which obtained compound is represented by formula (IV) 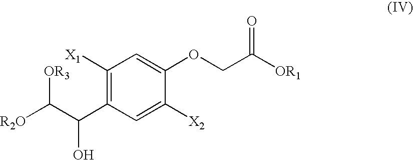
wherein R₁, R₂, R₃, X₁, and X₂ are as defined above into an aldehyde of formula (V) 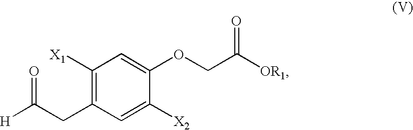
wherein R₁, X₁, and X₂ are as defined above by
i. transforming the free hydroxyl group of the compound of formula (IV) into a leaving group, and
ii. removing the previously generated leaving group under reductive conditions, and
C. reacting the aldehyde of formula (V) with 1-(4-hydroxy-phenyl)-1-hydroxy-2-propylamin or 4-hydroxy-norephedrine to produce the compound of formula (I).

2. The method according to claim 1, wherein R₁, R₂, R₃, X₁ and X₂ each represent a linear or branched C₁₋₃-alkyl group.

3. The method according to claim 1, wherein R₁ is ethyl, and wherein R₂, R₃, X₁, and X₂ each are methyl.

4. The method according to claim 1, wherein the Z is a halogen and step A is performed in the presence of catalytic amounts of sodium iodide.

5. The method according to claim 1, wherein the leaving group of the step B is a trialkylsilyloxy group.

6. The method according to claim 1, wherein the step C is carried out in the presence of a reducing agent.

7. The method according to claim 6, wherein the reducing agent is selected from the group consisting of alkali metal borohydrides, borane compounds, and hydrogen atmosphere in the presence of a metallic catalyst.

8. The method according to claim 1, wherein the step C involves addition or presence of an acid.

9. The method according to claim 1, wherein the compound of formula (I) is Ethyl (−)-2-[4-[2-[[(1S,2R)-2-hydroxy-2-(4-hydroxyphenyl )-1-methylethyl]-amino]-ethyl]-2,5-dimethylphenoxy]acetate hydrochloride.

10. The method according to claim 1,
wherein R₁ is ethyl, R₂, R₃, X₁, and X₂ are each methyl, and Z represents a chlorine or bromine atom, and
wherein the step A is carried out in the presence of a base and, optionally, catalytic amounts of sodium iodide, and
wherein the hydroxyl group of the compound of general formula (IV) is converted into a trimethylsilyloxy group as leaving group, and
wherein the aldehyde according to formula (V) is dissolved in THF and coupled with HNE under a hydrogen atmosphere and in the presence of PC/C with 4-hydroxy-norephedrine, using a solution comprising HCl in 1,4-dioxane for the work-up.

11. The method of claim 11, further comprising
D. recrystallizing the product obtained by step C using a mixture of ethanol and methyl tert-butyl ether as a solvent.

12. The method of claim 1, further comprising
D. recrystallising the product obtained by the step C, or
E. transforming the product obtained by the step C into a pharmacologically acceptable salt form as the hydrochloride.

13. The method of claim 12, further comprising
F. transforming the product obtained by the step D into a pharmacologically acceptable salt form as the hydrochloride.

14. A method for the preparation of compounds of general formula (V) 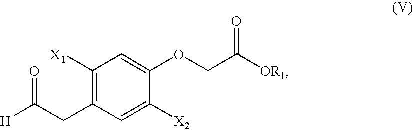
wherein R₁ is branched or unbranched C₁₋₆-alkyl, or optionally substituted benzyl, and wherein X₁ and X₂ are independently from each other hydrogen, halogen, or branched or unbranched C₁₋₆-alky, comprising the following steps:
A. transforming the free hydroxyl group of a compound of formula (IV) 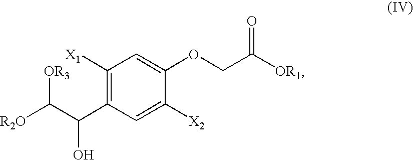
wherein R₁, X₁ and X₂ are as defined above, and wherein R₂ and R₃ are independently from each other a linear or branched C₁₋₆-alkyl group, into a leaving group; and
B. removing the previously generated leaving group under reductive conditions.

15. The method according to claim 14, wherein R₁, X₁, X₂, R₂ and R₃ independently from each other represent a linear or branched C₁₋₃-alkyl group.

16. The method according to claim 14, wherein R₁ is ethyl, and wherein X₁, X₂, R₂, and R₃ are each methyl.

17. The method according to claim 14, wherein the leaving group is a trialkylsilyloxy group.

18-20. (canceled)
